(19) United States
(12) Patent Application Publication
(10) Pub. No.: US 2009/0124909 A1
(43) Pub. Date: May 14, 2009

(54) Medical system and method for visualizing regions being monitored in a medical system

(76) Inventors: Uwe Danzer, Kalchreuth (DE); Stefan Sattller, Forchheim (DE); Reiner Staab, Baiersdorf (DE); Susanne Staab, Baiersdorf (DE); Katharina Staab, Aschaffenburg (DE); Silvia Rachor, Goldbach (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/284,589

(22) Filed: Sep. 23, 2008

(30) Foreign Application Priority Data
Sep. 24, 2007 (DE) 10 2007 045 528.5

(51) Int. Cl. A61B 6/00

(52) U.S. Cl. 600/476

(57) ABSTRACT

The invention relates to a medical diagnostic or therapy system comprising a system controller and at least one moving apparatus component, comprising a contour detecting unit which is constructed for detecting the position of components of the diagnostic or therapy system, and at least one projector that is decoupled from the contour detecting unit and is constructed for visualizing the region being monitored in which the component of the diagnostic and/or therapy system scanned by the contour detecting unit is arranged. The invention also relates to a method for visualizing regions being monitored in a medical diagnostic or therapy system of this kind.

### CROSS REFERENCE TO RELATED APPLICATIONS

This application claims priority of German application No. 10 2007 045 528.5 filed Sep. 24, 2007, which is incorporated by reference herein in its entirety.

### FIELD OF THE INVENTION

The invention relates to a medical diagnostic and/or therapy system and to a method for visualizing regions being monitored in a medical diagnostic and/or therapy system according to the claims.

### BACKGROUND OF THE INVENTION

Laser scanners have not previously been used in medical diagnostic and/or therapy systems to monitor moving apparatus components, such as a C-arm for example, because display of the regions being monitored by laser scanners is possible to only a very limited extent. With laser scanners, which work with lasers that are beyond the human field of perception, a direct display of the regions being monitored by way of the laser is not visible on the one hand, and an indirect method of distinguishing the laser beams, for example by infrared indicators or transducers in the beam path, means a limitation of the region being monitored on the other hand.

Laser scanners that operate with lasers in the visible wavelength range can in principle be perceived by the human eye. However even in this case there is the problem that rays of light that freely propagate in a space are not visible unless they strike a reflective surface (for example dust or smoke) or the beam is looked at directly. Both of these should be avoided in a medical environment.

In theory the application of markings on the floor would be possible but this is often also undesirable in a clinical environment.

### SUMMARY OF THE INVENTION

The object of the present invention is to provide a medical diagnostic and/or therapy system which allows use of a laser scanner for monitoring moving components of the diagnostic and/or therapy system. A further object of the invention is to provide a method that is suitable for use of the diagnostic and/or therapy system.

The object is achieved according to the invention by a medical diagnostic and/or therapy system and by a method according to the independent claims. Advantageous embodiments of the invention are the respective subject of the associated dependent claims.

The invention ensures that moving parts of a medical diagnostic and/or therapy system are monitored by means of a contour detecting and processing unit, such as in particular a laser scanner, and that with little effort and without limiting monitoring for the hospital staff, the regions being monitored are simultaneously rendered visible by means of a projector, in particular by projection onto the floor. Use of a laser scanner in the medical environment is in particular easily made possible.

The basic idea of the invention lies in using a projector that is decoupled from the contour detecting unit to display the regions being monitored and sources of disturbance. Consequently it is possible to use a laser scanner, which has previously always been critical primarily due to the invisible laser, in the medical field for the first time. “Decoupled” is in this case taken to mean that the projector is a self-contained, independently operable unit with its own light source but which can by all means be controlled by the same control unit as the contour detecting unit.

The contour detecting unit is advantageously formed by a laser scanner. Three-dimensional, primarily also moving, structures can be detected by means of the laser scanner with a high level of accuracy and, owing to the high speed of the laser scanner, can also be detected quickly. A laser scanner generally comprises a scanning head with laser and changeable mirrors, by way of which a laser beam scans surfaces or bodies line-wise or raster-wise, and an electronic driving and control device.

According to one embodiment of the invention the projector is formed by a video projector. Projections of a video projector can be projected on surfaces such as the floor so as to be particularly easily visible.

For simple and easily visible visualization and optical display the projector is advantageously constructed on the floor of the space in which the diagnostic and/or therapy system is arranged for projection of the region being monitored, in which the scanned part of the diagnostic and/or therapy system is disposed. For this purpose the projector is assembled in the examination room in such a way that unhindered projection onto the floor is possible. A plurality of projectors may also be provided. Projection onto any desired locations of the floor may thus be particularly easily ensured even if one of the projectors is hindered in direct projection owing to an obstacle. In the case of obstacles that shift the optical path length mirrors may also be provided which allow indirect projection onto the floor.

According to a further embodiment of the invention the diagnostic and/or therapy system comprises a control device which is constructed for joint control of the contour detecting unit and the projector. In particular it is provided that the system controller of the diagnostic and/or therapy system assumes control. Particularly realtime control of the two units is possible hereby. Respectively adjusted warning ranges can be set for both the contour detecting unit and the projector, for example as a function of the positions of moving components or as a function of the type of medical examination.

According to a further embodiment of the invention the diagnostic and/or therapy system is formed by an X-ray system with a moving C-arm.

According to the invention, in a method for visualizing regions being monitored in the medical diagnostic and/or therapy system a contour detection of at least one part of the diagnostic and/or therapy system is carried out by a contour detecting unit in a region being monitored, and realtime optical display of the region being monitored is carried out by a projector that is decoupled from the contour detecting unit.

Optical display advantageously takes place in realtime for uninterrupted progression of an examination by means of the diagnostic and/or therapy system. The region being monitored is expediently projected onto the floor of the space in which the diagnostic and/or therapy system is arranged.

### BRIEF DESCRIPTION OF THE DRAWINGS

The invention and further advantageous embodiments according to features of the subclaims will be described in more detail hereinafter with reference to schematically illustrated exemplary embodiments in the drawings, without this constituting a limitation of the invention to these exemplary embodiments. In the drawings:

FIG. 1 shows a view of a medical diagnostic system according to the invention;

FIG. 2 shows a control diagram between a projector, a control unit and a contour detecting unit.

### DETAILED DESCRIPTION OF THE INVENTION

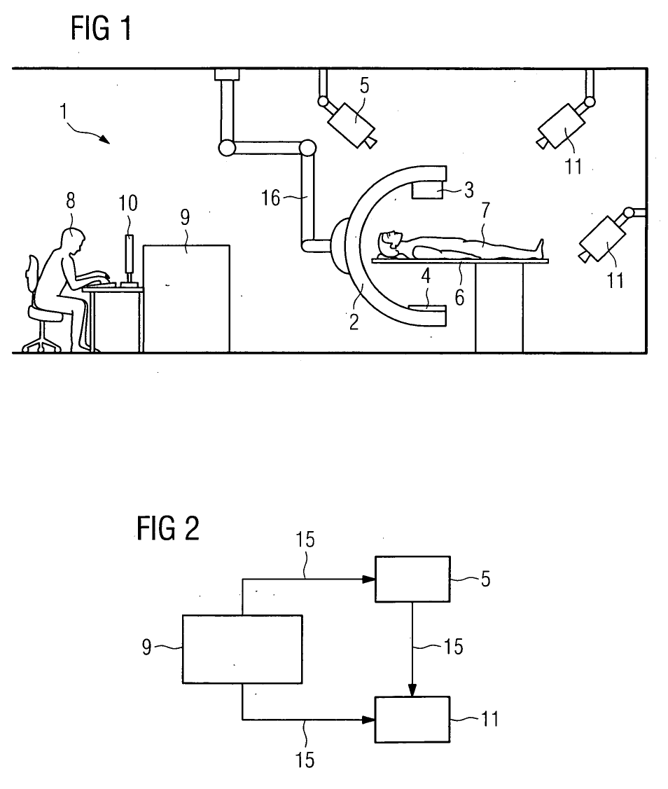

FIG. 1 shows a medical diagnostic system 1 according to the invention. The medical diagnostic system 1 comprises a moving C-arm 2, a system controller 9 that controls the medical diagnostic system, and a patient couch 6. An X-ray source 3 for emitting X-ray radiation and an X-ray detector 4 for detecting X-ray radiation are arranged on the C-arm 2. The C-arm 2 can be moved in several directions for example by a ceiling support on a moving arm 16. The system controller 9 is used to control the components of the medical diagnostic system 1 and can be operated by an operator 8 for example via a user interface or an operating panel.

According to the invention the diagnostic system 1 comprises a laser scanner 5 and at least one, in the illustrated case, two, video projector(s) 11. The laser scanner 5 is arranged in the examination room in such a way that scanning of the C-arm 2 and/or the patient couch 6 is possible without limitation. The two video projectors 11 are also arranged in the examination room in such a way that they can project any desired region being monitored onto the floor of the examination room.

Irrespective of the chosen application of the medical diagnostic system 1, the system controller 9 controls the laser scanner 5 for scanning part of the C-arm 2 or all of the C-arm 2 for example. During scanning of the C-arm 2 one of the video projectors 11 projects a corresponding colored or patterned area, which represents the region being monitored, onto the floor beneath the C-arm 2. The medical staff can thus distinguish the region being monitored scanned by the laser scanner 5. The projection corresponds to the currently active region being monitored by the laser scanner 5. An operator 8 can thus immediately clearly distinguish which regions are currently being monitored and whether there are dangers therein.

Coloration of the regions can be set by the user as desired. Different monitoring fields of the laser scanner 5 can be activated by the system controller software as a function of the positions of C-arm 2 and patient couch 6 or the type of medical examination. A warning function can also be triggered in the event of a critical movement registered by the laser scanner or an object that suddenly appears in the region being monitored. In this case the site of the disturbance can be emphasized by a special signal, for example a specific color, a specific pattern or flashing, in order to make the potential disturbance even more easily visible and identifiable to the operators. An acoustic signal can also be provided.

FIG. 2 schematically shows a control diagram. The system controller 9 controls the region being monitored of the laser scanner 5. For this the system controller 9 is connected to the laser scanner 5 by means of a bidirectional communications line 15. At the same time the system controller 9 controls the video projector 11 for displaying the regions being monitored on the floor of the examination room. A bidirectional communications line 15 is likewise provided between the system controller 9 and the video projector 11 for this purpose. It may also be provided that the laser scanner 5 directly informs the video projector 11 of the region being monitored, which it is monitoring, by means of a communications line 15.

As a result of the fact that the region being monitored and/or the disturbance is/are imaged by one or more video projector(s) and not by the laser scanner itself, the laser scanner can function unrestrictedly and without interruption. This kind of projection by a projector can also be dynamically adapted to any desired regions being monitored.

The invention may be briefly summarized as follows: for improved monitoring of moving components and dynamic display of the regions being monitored a medical diagnostic and/or therapy system with a system controller and at least one moving apparatus component is provided, comprising a contour detecting unit, which is constructed for detecting the position of parts of the diagnostic and/or therapy system, and at least one projector that is decoupled from the contour detecting unit and is constructed for visualizing the region being monitored, in which the part of the diagnostic and/or therapy system scanned by the contour detecting unit is arranged.

1-14. (canceled)

15. A medical system, comprising:
a moving component;
a contour detecting unit that detects a position of the moving component; and
a projector that is decoupled from the contour detecting unit and visualizes a region being monitored in which the moving component is arranged.

16. The medical system as claimed in claim 15, further comprising a control device that controls the contour detecting unit and the projector.

17. The medical system as claimed in claim 15, wherein the contour detecting unit is a laser scanner.

18. The medical system as claimed in claim 15, wherein the projector is a video projector.

19. The medical system as claimed in claim 15, further comprising a system controller that controls the medical system.

20. The medical system as claimed in claim 19, wherein the system controller controls the contour detecting unit and the projector.

21. The medical system as claimed in claim 15, wherein the projector projects the region being monitored on a floor of an examination room in which the medical system is arranged.

22. The medical system as claimed in claim 15, wherein the medical system comprises a plurality of projectors.

23. The medical system as claimed in claim 15, wherein the moving component is a patient couch.

24. The medical system as claimed in claim 15, wherein the medical system is a medical diagnostic or therapy system.

25. The medical system as claimed in claim 15, wherein the medical system is an X-ray system and the moving component is a moving C-arm of the X-ray system.

26. A method for displaying a region being monitored in a medical system, comprising:
contour detecting a position of a part of the medical system arranged in the region being monitored by a contour detecting unit;
providing a projector that is decoupled from the contour detecting unit; and
displaying the region being monitored by the projector.

27. The method as claimed in claim 26, wherein the contour detecting unit and the projector are controlled by a control device.

28. The method as claimed in claim 26, wherein the region being monitored is displayed in realtime.

29. The method as claimed in claim 26, wherein the region being monitored is optically displayed.

30. The method as claimed in claim 26, wherein a part of data obtained during the contour detection is used in the display.

31. The method as claimed in claim 26, wherein the region being monitored is projected on a floor of an examination room in which the medical system is arranged.

32. The method as claimed in claim 26, wherein the contour detecting unit and the projector are controlled by a system controller of the medical system.

33. The method as claimed in claim 26, wherein the part of the medical system comprises a moving component of the medical system.
